(19) United States
(12) Patent Application Publication
(10) Pub. No.: US 2013/0123627 A1
(43) Pub. Date: May 16, 2013

(54) OBJECT INFORMATION ACQUIRING APPARATUS AND CONTROL METHOD THEREOF

(71) Applicant: Canon Kabushiki Kaisha, Tokyo (JP)

(72) Inventor: Kenji Oyama, Tokyo (JP)

(73) Assignee: CANON KABUSHIKI KAISHA, Tokyo (JP)

(21) Appl. No.: 13/666,309

(22) Filed: Nov. 1, 2012

(30) Foreign Application Priority Data
Nov. 10, 2011 (JP) 2011-246414

(51) Int. Cl. A61B 8/08; A61B 8/14

(52) U.S. Cl. 600/442

(57) ABSTRACT

Disclosed is an object information acquiring apparatus for acquiring object information, including: a probe including a plurality of elements arranged along at least a first direction and configured to sequentially perform transmitting of acoustic wave beams and receiving of reflected waves along the first direction by the plurality of elements; a scanning unit configured to set a second direction intersecting the first direction as a main scanning direction and move the probe at a predetermined speed; and a adjusting unit configured to acquire information on a measurement depth for acquiring object information in a transmitting direction of the acoustic wave beams and determine the number of times of transmitting of acoustic wave beams and receiving of reflected waves along the first direction based on the depth, resolution of the object information in the main scanning direction, and a moving speed of the probe.

### BACKGROUND OF THE INVENTION

1. Field of the Invention

The present invention relates to an object information acquiring apparatus and a control method thereof.

2. Description of the Related Art

An ultrasound measuring apparatus that transmits an ultrasound wave to a living body and analyzes a reflected ultrasound wave to image an in vivo structure has been used in a medical field. When the ultrasound wave is transmitted to a living body, ultrasound reflection occurs at boundary surfaces in the living body which have different acoustic impedance. The ultrasound measuring apparatus may receive and analyze the reflected wave to obtain tissue information in the object.

The ultrasound measuring apparatus may recognize a position or a size of a tumor in a depth direction (a transmission direction of an ultrasound beam) as an image. In addition, since an acoustic measurement is performed using an ultrasound wave, an in vivo tissue may be measured non-invasively, which greatly reduces a physical burden of a patient.

In an ultrasound diagnosis apparatus having a function of setting a desired measurement depth and performing focusing, a technology of increasing a frame rate as maximally as possible is disclosed in Japanese Patent Application Laid-Open No. 2010-94171 (PTL 1: Patent Literature 1). The ultrasound diagnosis apparatus disclosed in Japanese Patent Application Laid-Open No. 2010-94171 determines an ultrasound transmitting and receiving interval and then controls transmission and reception of an ultrasound wave, based on a depth of a boundary position at the time of synthesizing signals between different focuses. As a result, it is possible to optimally set the ultrasound transmitting and receiving interval and more improve a frame rate than the case in which the measurement depth is uniform.

Further, in an apparatus for transmitting and receiving an ultrasound wave at a determined constant ultrasound transmitting period, a technology of validly using time of the transmitting period is disclosed in Japanese Patent Application Laid-Open No. H3-126442 (PTL 2: Patent Literature 2). According to a technology disclosed in Japanese Patent Application Laid-Open No. H3-126442, when the ultrasound transmitting period is not less than twice as long as the ultrasound transmitting and receiving period required for measurement, an interleave scanning is performed for an idle time in the transmitting period. Accordingly, it is possible to improve the frame rate by validly using measurement time.
PTL 1: Japanese Patent Application Laid-Open No. 2010-94171
PTL 2: Japanese Patent Application Laid-Open No. H3-126442

### SUMMARY OF THE INVENTION

In the ultrasound measuring apparatus according to the related art, it takes time to measure an object. For example, in mammography performing an examination of a breast cancer, a breast that is an object portion is pressurized and fixed for measurement but it is preferable to reduce a time to apply a burden on an object due to the pressurization.

The ultrasound measuring apparatus for generating three-dimensional ultrasound images configured by a plurality of tomographic images aligned at a predetermined interval needs to generate the tomographic images sheet by sheet according to a voxel pitch of targeted ultrasound images. That is, since the ultrasound measuring apparatus needs to sequentially acquire the ultrasound signals required to generate the tomographic images while seamlessly moving a probe, the ultrasound measuring apparatus needs to acquire the ultrasound signals corresponding to a sheet of tomographic image before the probe reaches a position at which a next tomographic image is acquired. Therefore, a moving speed of the probe at the time of measurement cannot be faster than a maximum speed meeting the above conditions.

Further, the acquisition time of the ultrasound signals is long since the deeper the targeted measurement depth, the longer the time to propagate an ultrasound wave becomes. That is, the deeper the measurement depth, the slower the moving speed of the probe becomes. To the contrary, when intending to secure the steady moving speed, the measurement depth is limited. Recently, a demand for high resolution of the ultrasound images is increased, but when intending to acquire the ultrasound signals at a finer pitch coping therewith, the measurable maximum depth is more limited since time allocated to process a sheet of tomographic image is short.

Here, the moving speed of the probe is considered. The moving speed of the probe is obtained by dividing the acquisition pitch of the ultrasound signals that can be calculated in the resolution of images by the time required to acquire the ultrasound signals that can be calculated in the measurement depth. That is, in the object information acquiring apparatus according to the related art, when the image resolution is uniform, the movement of the probe is slow if the measurable depth is set to be deep, and the movement of the probe is fast if the measurable depth is set to be shallow.

Here, the following problem may be derived.

A first problem is that a dead time is caused when the acquisition time of the ultrasound signals is short if the moving speed of the probe is set to be slow by making the measurable depth deep. That is, when the measurement depth of the three-dimensional ultrasound images is shallow, a redundant time for which the processing is not performed is caused.

A second problem is that when the moving speed of the probe is set to be fast by making the measurable depth shallow, the redundant time is removed, but a place at which it takes time to acquire the ultrasound signals cannot be measured. That is, when the measurement depth of the three-dimensional ultrasound image is deep, the time to perform the processing is insufficient.

It may be considered that the above problem may be resolved by varying the moving speed of the probe over the acquisition time of the ultrasound signals, that is, making the moving speed slow at a place where it takes time to acquire the signal and making the moving speed fast at a place where it takes less time to acquire the signal. However, the object information acquiring apparatus may hardly acquire the distance and the time for accelerating and decelerating the probe and may not easily control a speed since an acquisition pitch of the ultrasound signals is fine.

Further, in the case in which the ultrasound beams are transmitted and received while continuously moving the probe, since a slope of a section direction is changed and images are distorted when the moving speed of the probe is changed during the measurement, it is difficult to perform a comparison for each ultrasound image. For this reason, it is preferable to make the moving speed of the probe constant at all times during the measurement.

Both of the inventions disclosed in Japanese Patent Application Laid-Open No. 2010-94171 and Japanese Patent Application Laid-Open No. H3-126442 may improve real time capability to improve the frame rate at the time of acquiring the images, but do not consider the overall scanning time and therefore, cannot resolve the above problems.

In view of the problems, an object of the present invention is to provide an object information acquiring apparatus capable of providing a method of acquiring acoustic wave data appropriate for a measurement depth.

The present invention provides an object information acquiring apparatus comprising:

a probe including a plurality of elements arranged along at least a first direction and configured to sequentially perform transmitting of acoustic wave beams and receiving of reflected waves reflected from an inside of an object along the first direction by a part of or all of the elements;

a scanning unit configured to set a second direction intersecting the first direction as a main scanning direction and move the probe in the main scanning direction at a predetermined speed; and

an adjusting unit configured to acquire information on a measurement depth for acquiring object information in a transmitting direction of the acoustic wave beams and determine the number of times of transmitting of acoustic wave beams and receiving of reflected waves along the first direction based on the measurement depth, resolution of the object information in the main scanning direction, and a moving speed of the probe.

The present invention also provides a control method of an object information acquiring apparatus that includes an probe including a plurality of elements arranged along at least a first direction and configured to sequentially perform transmitting of acoustic wave beams and receiving of reflected waves reflected from an inside of an object along the first direction by a part of or all of the elements, and that is configured to set a second direction intersecting the first direction as a main scanning direction and move the probe in the main scanning direction at a predetermined speed to acquire object information, the method comprising the steps of:

acquiring information on a measurement depth for acquiring the object information in a transmitting direction of the acoustic wave beams; and

determining the number of times of transmitting of acoustic wave beams and receiving of reflected waves along the first direction based on the measurement depth, resolution of the object information in the main scanning direction, and a moving speed of the probe.

According to the embodiment of the present invention, it is possible to provide an object information acquiring apparatus capable of providing a method of acquiring acoustic wave data appropriate for a measurement depth.

Further features of the present invention will become apparent from the following description of exemplary embodiments with reference to the attached drawings.

### BRIEF DESCRIPTION OF THE DRAWINGS

FIG. 1 is a diagram showing a system configuration of an ultrasound measuring apparatus according to a first embodiment;

FIGS. 2A and 2B are conceptual diagrams for explaining a method of measuring ultrasound waves according to the first embodiment;

FIGS. 3A and 3B are diagrams showing a relationship between electronic scanning and a signal acquisition time in a maximum depth according to the first embodiment;

FIG. 4 is a conceptual diagram for explaining in detail an electronic scanning method according to the first embodiment;

FIGS. 5A and 5B are diagrams for explaining a method of acquiring ultrasound data in the maximum depth according to the first embodiment;

FIGS. 6A and 6B are diagrams showing a relationship between the electronic scanning and the signal acquisition time when the depth is deep, according to the first embodiment;

FIGS. 7A and 7B are diagrams for explaining the method of acquiring ultrasound data when the depth is deep, according to the first embodiment;

FIG. 8 is a flow chart showing a flow of acquiring the ultrasound data according to the first embodiment;

FIGS. 9A and 9B are diagrams showing a relationship between electronic scanning and a signal acquisition time when the depth is shallow, according to a second embodiment; and

FIGS. 10A and 10B are diagrams for explaining a method of acquiring ultrasound data when the depth is shallow, according to the second embodiment.

### DESCRIPTION OF THE EMBODIMENTS

### First Embodiment

Hereinafter, a first embodiment of the present invention will be described with reference to the accompanying drawings.

An object information acquiring apparatus according to the first embodiment is an ultrasound measuring apparatus using an ultrasound echo technology of transmitting acoustic wave beams, that is, ultrasound beams to an object and receiving waves reflected from an inside of the object to acquire object information as image data. The acquired object information is information reflecting a difference in acoustic impedance of an internal tissue of an object.

Further, in describing embodiments, a main scanning direction means a direction in which a probe acquires ultrasound signals while being moved, and a sub-scanning direction means a direction intersecting the main scanning direction. In addition, ultrasound data means all the data required to generate three-dimensional ultrasound images that are acquired from an area to be measured. Further, the ultrasound signal means a signal generated by allowing one or a plurality of elements (a part of or all of the elements) to receive reflected waves. In addition, an ultrasound beam means a set of ultrasound waves which are transmitted by shifting phases thereof so as to converge the ultrasound waves at a specific point by the plurality of elements. Further, an electronic scanning width means a width along the sub-scanning direction in which ultrasound beams for measurement are transmitted.

Further, as the scanning performed by the object information acquiring apparatus, there are two types, that is, mechanical scanning mechanically moving the probe on a two-dimensional plane and electronic scanning transmitting and receiving ultrasound beams generated by the plurality of elements while moving the ultrasound beams in the sub-scanning direction. In describing embodiments, the former is referred to as probe scanning and the latter is referred to as ultrasound scanning.

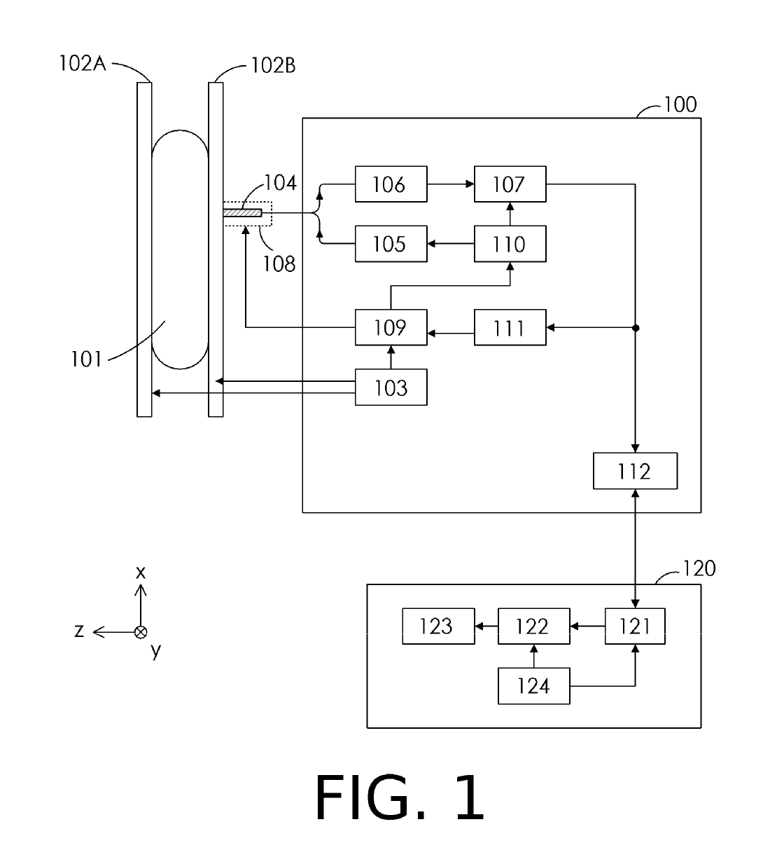

FIG. 1 is a diagram showing a system configuration of an ultrasound measuring apparatus according to the first embodiment.

The ultrasound measuring apparatus according to the first embodiment largely includes a measuring apparatus 100 and an image processing apparatus 120. The measuring apparatus 100 is an apparatus for performing a measurement of an object using an ultrasound wave, and the image processing apparatus 120 is an apparatus for operating the measuring apparatus 100 and visualizing measured data. The measuring apparatus 100 includes a holding plate 102, a holding control unit 103, a probe 104, an ultrasound transmitting unit 105, an ultrasound receiving unit 106, a signal processing unit 107, a moving mechanism 108, a moving control unit 109, a scanning control unit 110, and an interface 112.

The image processing apparatus 120 includes an interface 121, an image construction unit 222, a display unit 223, and an operation unit 124. Generally, an apparatus having a high-performance arithmetic processing function or a graphic display function such as a PC, a workstation, and the like, is used. Hereinafter, an object measuring method will be described while describing each component.

First, a configuration of the measuring apparatus 100 is described.

As an object 101, a human body, in detail, portions to be diagnosed, such as a breast, a finger, a hand, a foot, and the like, of a human or an animal, are considered. The object 101 is fixed to holding plates 102A and 102B fixing an inspection unit to an apparatus in a form interposed into both sides thereof.

The holding plate 102 is a holding member that constantly maintains a shape of at least a part of the object and is mounted between an object and a probe and is formed in a pair of two sheets of 102A and 102B. The object is interposed into both sides of the holding members and a position thereof is fixed during measurement, such that a position error thereof due to a body motion, and the like, may be reduced. Further, an ultrasound wave may efficiently reach a deep part of an object by the holding.

As the holding member, it is preferred to use a member having high acoustic matching capability with the object or the probe while having high propagation efficiency of an ultrasound wave. In particular, the holding plate 102B is positioned in a propagation path of an ultrasound wave and therefore, is preferably a member having high acoustic matching capability with the ultrasound probe. In order to increase the acoustic matching capability, an acoustic matching material such as a gel, and the like, may be preferably interposed between the holding plate and the object, and the holding plate and the probe. The holding plates are controlled to have a holding interval appropriate for measurement by the holding control unit 103. The holding plate 102A and the holding plate 102B are collectively marked as the holing plate 102 when there is no need to differentiate the holding plate 102A and the holding plate 102B.

The holding control unit 103 controls a holding state of the object 101 at the holding interval and a holding pressure appropriate for the ultrasound measurement so as to meet a burden or a measurement depth of an object. Further, the holding control unit 103 controls the holding state of the object to be constantly maintained during the measurement of an ultrasound wave. In addition, the holding information (maintenance distance and holding pressure) of the object is output to the moving control unit 109 at the time of measuring the ultrasound waves. In the present invention, the measurement depth is a distance of a depth direction (a transmitting direction of an ultrasound beam) for acquiring object information. In the first embodiment, adding the maintenance distance to a thickness of the holding plate 102B is defined as the ultrasound measurement depth.

The probe 104 is a means that is configured by arranging an ultrasound source and a plurality of elements, and transmits ultrasound beams to the object and receives an ultrasound echo reflected from an inside of the object to convert the received ultrasound echo into an electrical signal. As a general ultrasound probe, a conversion element using piezoelectric ceramics (PZT), a capacitive microphone conversion element, and the like, are used.

Further, a capacitive micromachined ultrasound transducer (CMUT), a magnetic MUT (MMUT) using a magnetic film, and the like, may also be used. In addition, as the ultrasound probe, any type, such as a piezoelectric MUT (PMUT) using a piezoelectric thin film, and the like, may be used.

Further, in the ultrasound measuring apparatus performing measurement by moving the probe while the probe contacting the holding plate 102B having a two-dimensional plane shape, a linear scanning type probe capable of generating tomographic images having uniform image quality in parallel ultrasound beams is generally used. In the first embodiment, for explanation, an example of using a one-dimensional probe in which the elements are linearly arranged in a row will be described below. However, the object information acquiring apparatus according to the present invention may be configured to perform the measurement using a two-dimensionally arranged array type probe (also including a 1.5 D probe). In addition, in the description of the embodiment, the movement of the ultrasound beams is realized by switching an electronic switch, and the like, and therefore, the ultrasound scanning is described using a term called electronic scanning.

The scanning control unit 110 generates driving signals applied to each element configuring the probe 104 to control a frequency and a sound pressure of the transmitted ultrasound wave. In addition, the scanning control unit 110 includes a transmitting control function of setting a transmitting direction of ultrasound beams to select a transmitting delay pattern corresponding to the transmitting direction and a receiving control function of setting a receiving direction of ultrasound signals to select a receiving delay pattern corresponding to the receiving direction. The transmitting delay pattern is a pattern of a delay time allocated to the plurality of driving signals so as to form the ultrasound beams in a predetermined direction by the ultrasound waves transmitted from a part of or all of the plurality of elements. In addition, the receiving delay pattern is a pattern of a delay time allocated to a plurality of receiving signals so as to extract ultrasound signals from any direction of the ultrasound signals detected by a part of or all of the plurality of elements. These transmitting delay patterns and the receiving delay patterns are stored in a separate memory means (not illustrated).

The ultrasound transmitting unit 105 applies the driving signals generated by the scanning control unit 110 to individual elements configuring the probe 104.

The ultrasound receiving unit 106 includes a signal amplifying unit that amplifies analog signals detected by a plurality of elements configuring the probe 104 and an A/D conversion unit that converts an analog signal into a digital signal to convert a received signal into a digital signal.

The signal processing unit 107 performs receiving focus processing on the signal generated by the ultrasound receiving unit 106 by adding each signal corresponding to each delay time, based on the receiving delay pattern selected by the scanning control unit 110. The ultrasound signals having a narrow focus are generated by the processing. Further, the signal processing unit 107 performs a time gain control (TGC), and the like, that increases and decreases an amplification gain according to the depth of the reflected position of the ultrasound wave so as to generate the tomographic images having uniform contrast without depending on the measurement depth.

In addition, in the first embodiment, if the ultrasound signals may finally generate the tomographic images of a B mode, any type of ultrasound signals may be used. For example, the ultrasound signals may be envelope data subjected to envelope detection processing using a low-pass filter, and the like, or data obtained by performing processing such as logarithmic compression, gain adjustment, and the like, on the envelope data.

The moving mechanism 108 includes a driving unit such as a motor, and the like, and mechanical parts transferring the driving force and is a driving mechanism receiving an order of the moving control unit 109 to move the probe 104 on the holding plate 102B. In addition, the moving mechanism 108 detects position information of the probe 104 and outputs the detected position information to the moving control unit 109.

The moving control unit 109 controls the moving mechanism 108 so as to two-dimensionally move the probe 104 on the holding plate. In addition, when the probe 104 reaches an acquisition start position of the ultrasound signal, an acquisition order of the ultrasound signal is issued to the scanning control unit 110. It is possible to obtain a wide measurement area by two-dimensionally moving the probe 104, and for example, it is possible to acquire ultrasound data in a full breast at the time of diagnosing a breast cancer. In addition, the moving control unit 109 calculates the measurement depth and the ultrasound transmitting and receiving time and performs the adjustment of the electronic scanning width of ultrasound beams and shifted amount of probe scanning, based on the holding information received from the holding control unit 103. The detailed operation thereof will be described below.

A control unit 111 receives a measuring start order or various demands from the image processing apparatus 120 to manage and control the overall ultrasound measuring apparatus. In addition to transferring the measuring start to the scanning control unit 110, the control unit 111 serves to manage identification information for identifying an individual apparatus or information peculiarly set in each apparatus, monitor an apparatus state, transfer the information to the image processing apparatus 120, and the like.

The interface 112 is an input and output means that transmits the apparatus information to the image processing apparatus 120 together with the ultrasound data and receives various orders from the image processing apparatus 120. The interface 112 serves to perform data communication between the measuring apparatus 100 and the image processing apparatus 120, together with the interface 121 of the image processing apparatus 120. It is preferable to adopt a communication protocol which can secure real time capability and implement large-capacity transmission.

Next, a configuration of the image processing apparatus 120 is described.

The interface 121 has the same function as the interface 112 of the ultrasound measuring apparatus and transmits ultrasound data, various orders for an apparatus, and the like, in two ways, together with the interface 112.

The image construction unit 222 images the tissue information within the object and constructs three-dimensional ultrasound images, based on the transmitted ultrasound data. Further, the image construction unit 222 may have a function of constructing the ultrasound images in a more preferable shape for diagnosis by applying various correction processings, such as adjustment or distortion correction of brightness, excision of an attractive area, and the like, to the constructed ultrasound images.

Further, the image construction unit 222 serves to adjust parameters for the construction of ultrasound images, displayed images, and the like, according to an operation of an operation unit 224 by a user. In addition, it is preferable to match a voxel pitch (resolution) of ultrasound images to be displayed with an acquisition pitch of the ultrasound signals. The reason is that extra interpolation processing and the like may be omitted and the acquired ultrasound signal may be most effectively used.

The display unit 223 is a display apparatus that displays three-dimensional ultrasound images constructed by the image construction unit 222. Further, the operation unit 224 is an input device for a user to perform the operations of the apparatuses such as designation of a measured position, adjustment of measurement, and the like, or an image processing operation for ultrasound images using operation software (not illustrated) of the ultrasound measuring apparatus.

The ultrasound apparatus according to the present embodiment may have the foregoing configuration to acquire the ultrasound data appropriate for the measurement depth and provide the three-dimensional ultrasound images to a user. Further, FIG. 1 shows that the image processing apparatus 120 is an external apparatus and the ultrasound measuring apparatus and the image processing apparatus are configured in separate hardware. However, the ultrasound measuring apparatus and the image processing apparatus may be integrally configured by aggregating each function.

(Details of Scanning Method Using Probe)

Next, a method of performing scanning of an object by the probe will be described below.

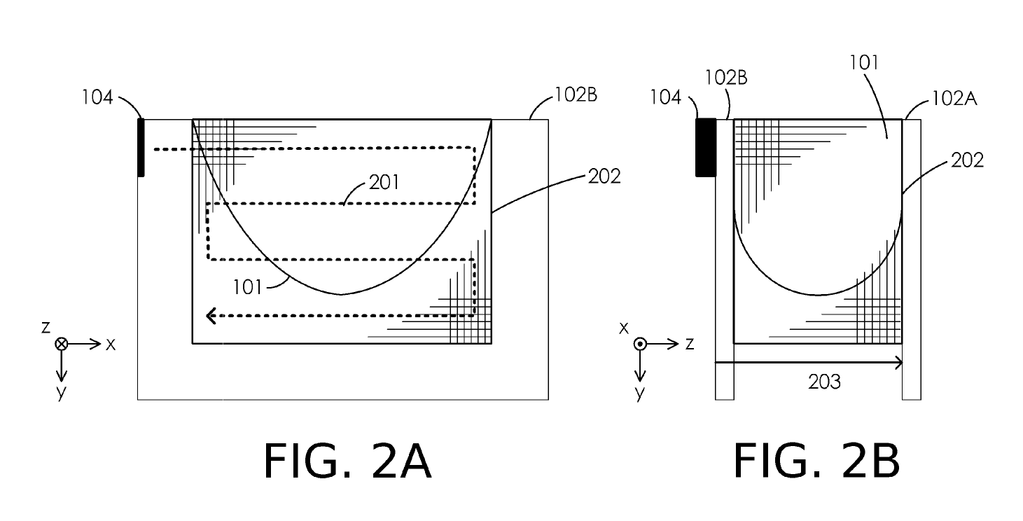

FIG. 2 is a conceptual diagram for explaining a measuring method on a two-dimensional plane using the ultrasound probe according to the first embodiment. FIG. 2A is a front view of the held object 101 viewed from the holding plate 102B with which the probe is in contact, and FIG. 2B is a side view of the held object 101. Reference numeral 201 shown by a dotted line represents a moving trajectory of the ultrasound probe and reference numeral 202 represents a measured data range obtained by the two-dimensional scanning. The acquisition area of data may be randomly set by a user.

The measured data are acquired by repeatedly performing main scanning for acquiring the ultrasound signals according to the acquisition pitch of the ultrasound signals while the probe is moved in an x-axis direction along the moving trajectory 201 and sub-scanning for moving the probe in a y-axis forward direction as much as a predetermined distance. Further, in the present invention, the main scanning direction (x-axis direction) is a second direction and in the present invention, a sub-scanning direction (y-axis direction) intersecting the main scanning direction is a first direction. Further, in the following description, a plane image that may be acquired from one-time electronic scanning and may be acquired from a y-z axis plane in FIG. 2 is referred to as the tomographic image.

A plurality of sheets of tomographic images may be acquired by scanning the probe in the main scanning direction (x-axis direction), and the three-dimensional ultrasound images using the electronic scanning width as a width in the y-axis direction may be acquired by arranging the acquired tomographic images along the x-axis. The three-dimensional ultrasound images having a targeted size are generated by performing the repeated scanning while the probe is moved in the y-axis direction by a predetermined distance and coupling the plurality of acquired ultrasound images.

The measured data acquired from the measured data range 202 are configured by data aligned based on the voxel pitch appropriate for image diagnosis. In a data pitch of each axis, such as an x axis, a y axis, and a z axis, for example, a pitch in an x-axis direction is a reciprocal number of resolution of the ultrasound image, a pitch in a y-axis direction is a distance between neighbor ultrasound beams transmitted from the probe 104, and a data pitch in a z-axis direction is a value in proportion to a sampling period of the ultrasound signals.

In the first embodiment, as shown in FIG. 2B, a measurement depth 203 is defined as a sum of a maintenance distance of the object 101 and a distance of the holding plate 102B thickness.

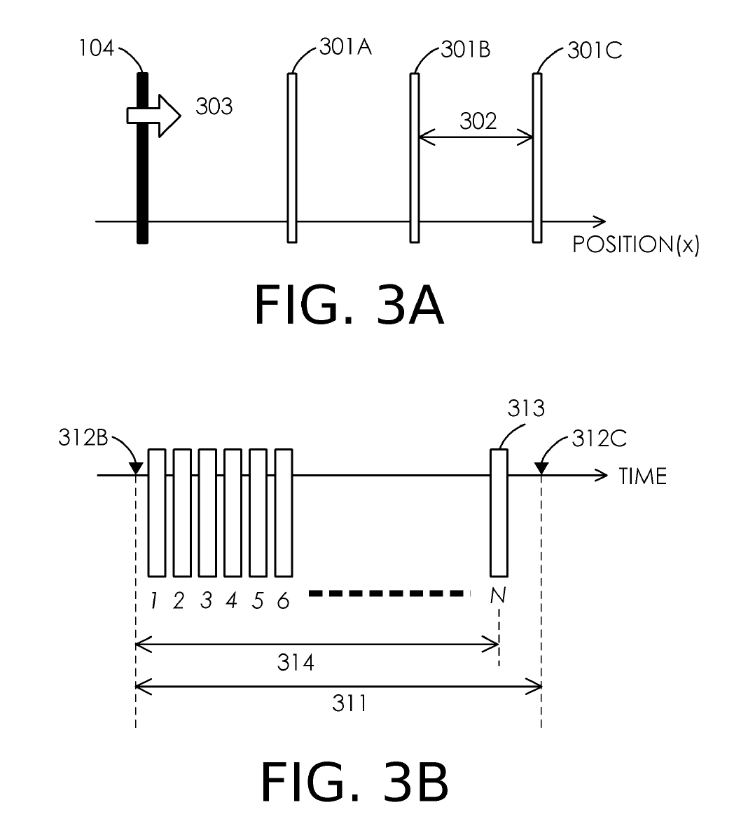

A relationship between the acquisition pitch of the ultrasound signals and the signal acquisition time according to the electronic scanning is described with reference to FIG. 3. FIG. 3A illustrates a position relationship between the movement of the probe 104 in the main scanning direction and the acquisition pitch of the ultrasound signals, and FIG. 3B illustrates a time relationship between the movement of the probe and the acquisition time of the ultrasound signals.

Reference numerals 301A, 301B, and 301C represent the acquisition start positions of the ultrasound signals, and an interval of reference numerals 301A to 301C is an acquisition pitch 302 in the x-axis direction of the ultrasound signal corresponding to the single tomographic image. The acquisition start position of the ultrasound signal is referred to as a signal acquisition start position hereinafter. The probe 104 starts the electronic scanning at positions of reference numerals 301A to 301C while moving in the main scanning direction at a constant moving speed 303. At each point of reference numerals 301A to 301C, the probe 104 transmits a predetermined number of times of transmitting of ultrasound beams and receiving of reflected waves and acquires the reflected waves for all the ultrasound beams transmitted to the next point, by using the plurality of elements disposed in the sub-scanning direction.

The two-dimensional tomographic images are acquired sheet by sheet at each point of reference numerals 301A to 301C by sequentially transmitting and receiving the ultrasound beams in the sub-scanning direction. The acquisition pitch 302 may be obtained by taking a reciprocal number of resolution of the object information to be acquired, that is, resolution of the ultrasound images. The number of transmitting of ultrasound beams and receiving of reflected waves is previously defined for each apparatus and may be increased and decreased as needed. The predetermined number is the reference number of transmitting of acoustic wave beams and receiving of reflected waves in the present invention. The detailed description thereof will be described below.

Reference numerals 312B and 312C each represent an acquisition start time of the ultrasound signals on a time base, corresponding to the signal acquisition start position 301B and the signal acquisition start position 301C. An acquisition period 311 of the ultrasound signal for acquiring the pitch 302 is determined by dividing the pitch 302 by the moving speed 303. The electronic scanning needs to be completed within the time of reference numeral 311, that is, the reflected waves for all the transmitted ultrasound beams need to be acquired. In the present invention, the acquisition period 311 is a first time.

A transmitting and receiving time 313 represents a time required to transmit the ultrasound beams for measuring the measurement depth 203 once and receive the ultrasound signals, wherein a horizontal width corresponds to the transmitting and receiving time. Since in order to acquire a sheet of tomographic image by the electronic scanning, the plurality of ultrasound beams needs to be transmitted and the reflected waves corresponding to the transmitted ultrasound beams need to be received, the time required to perform the electronic scanning once is represented by a signal acquisition time 314. The signal acquisition time 314 is within the first time, that is, needs to be shorter than the period 311. Further, it is preferable to set a slight spare time as an operation time of the apparatus for acquiring the next ultrasound signal.

When the number of transmitting of ultrasound beams and receiving of reflected waves is N and the time required to transmit one ultrasound beam and then obtain a reflected wave corresponding to the ultrasound beam is t, the signal acquisition time 314 is a sum of t and therefore, may be represented by N×t. In the present invention, the signal acquisition time 314 is a second time.

The more detailed example will be described. When the number of transmitting of ultrasound beams and receiving of reflected waves is N, an in vivo speed of sound is vb, and the measurement depth is d, t=2d/vb, such that the signal acquisition time 314 may be represented by N×(2d/vb) . . . Equation (1).

In addition, when the acquisition pitch of the ultrasound signals that is the reciprocal number of the image resolution is L, and the moving speed of the probe is u, the period 311 may be represented by L/u . . . Equation (2).

That is, when the electronic scanning is performed, there is a need to meet the relationship of N×(2d/vb)≦(L/u) . . . Equation (3).

Here, a reference measurement depth will be described. The reference measurement depth is a value representing a maximum depth that may be compatible with the number of transmitting of ultrasound beams and receiving of reflected waves along the predetermined sub-scanning direction, the image resolution, and the moving speed of the probe in the apparatus and is a unique value for the measuring apparatus. That is, the maximum measurement depth d meeting Equation (3) is a reference measurement depth.

In addition, the reference measurement depth represents the maximum measuring depth appropriate for an apparatus and is not the maximum depth in the apparatus design. The holding control unit 103 can implement the holding interval of an object at the foregoing reference measurement depth or more in consideration of a state of an object such as a size and a hardness of a cyst in a breast, and the like or a burden of an object. Thereafter, the description will be continued by considering the measurement depth 203 as a reference measurement depth.

Here, when intending to measure the depth exceeding the reference measurement depth 203, the transmitting and receiving time 313 of the ultrasound beams is long and thus, the acquisition time 314 of the ultrasound signal may exceed the period 311. Further, when intending to increase the number of ultrasound beams without changing the measurement depth, the acquisition time 314 of the ultrasound signal may also exceed the period 311. In this case, the next ultrasound signal cannot be acquired according to the pitch 302 and therefore, the resolution of the ultrasound images cannot be maintained.

As described above, when the image resolution and the moving speed of the probe are fixed, it can be appreciated that there is the restriction relationship between the measurement depth and the number of transmitting of ultrasound beams and receiving of reflected waves along the sub-scanning direction (electronic scanning width) and there is a need to meet conditions therebetween.

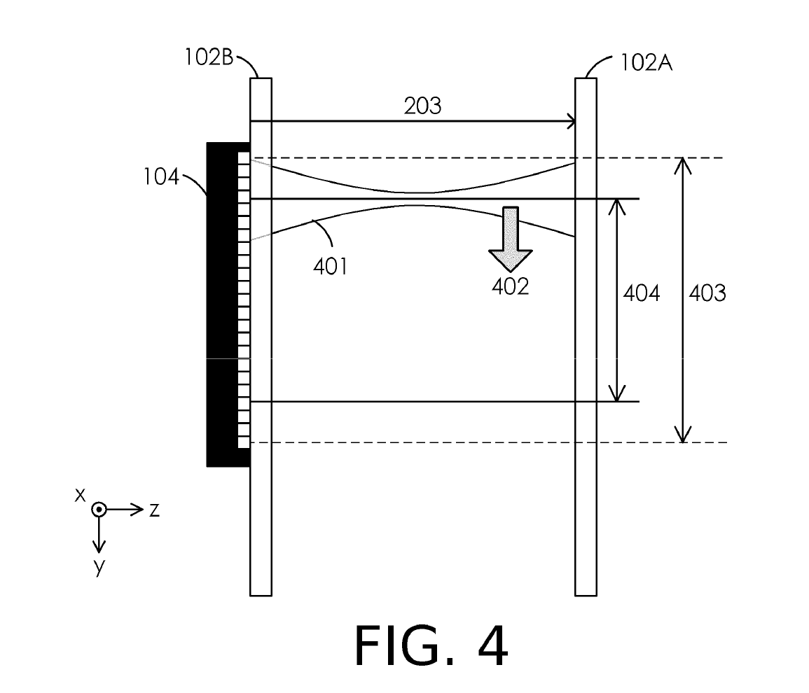

Next, the method of acquiring ultrasound signals will be described with reference to FIG. 4 showing the apparatus viewed from the side. FIG. 4 is a conceptual diagram for explaining the method of acquiring one ultrasound signal configuring the measurement data according to the first embodiment.

The probe 104 is configured of the plurality of elements aligned in a linear shape. An ultrasound beam 401 is formed by using a part of a plurality of element groups which are continuously arranged among the elements, and the electronic scanning is performed by moving the ultrasound beam 401 along a sub-scanning direction 402. It is possible to acquire the ultrasound signals required to generate the tomographic images having a width 403 approximately matched with the width of the probe 104 by performing the electronic scanning once.

However, since an aperture (a width of the element group) sufficient to form the ultrasound beams cannot be obtained at an end of the probe 104, the reliability of the acquired object information may be degraded, as compared with the case in which the sufficient aperture can be obtained. For this reason, it is preferable to acquire the ultrasound signals from a width 404 that can generally obtain the sufficient aperture, except for the case in which the electronic scanning using the end of the probe is unavoidable. In the diagnosis of the breast cancer holding and diagnosing a breast using the holding plate, the measurement of a base portion that is a body portion of a breast corresponds to an area in which the end of the probe is used.

For example, when the probe in which all the 128 elements are arranged at an element pitch at 0.25 mm are used, if the electronic scanning is performed by forming the ultrasound beam 401 using 32 elements, the width 403 is set to be 32 mm and the width 404 is set to be 24 mm. 16 elements from both ends of the probe, that is, 4 mm from both ends becomes an area in which the sufficient aperture cannot be obtained. Therefore, it is preferable that the width in which the electronic scanning is performed is set to be 24 mm as an upper bound so as to obtain the aperture enough to form the ultrasound beams.

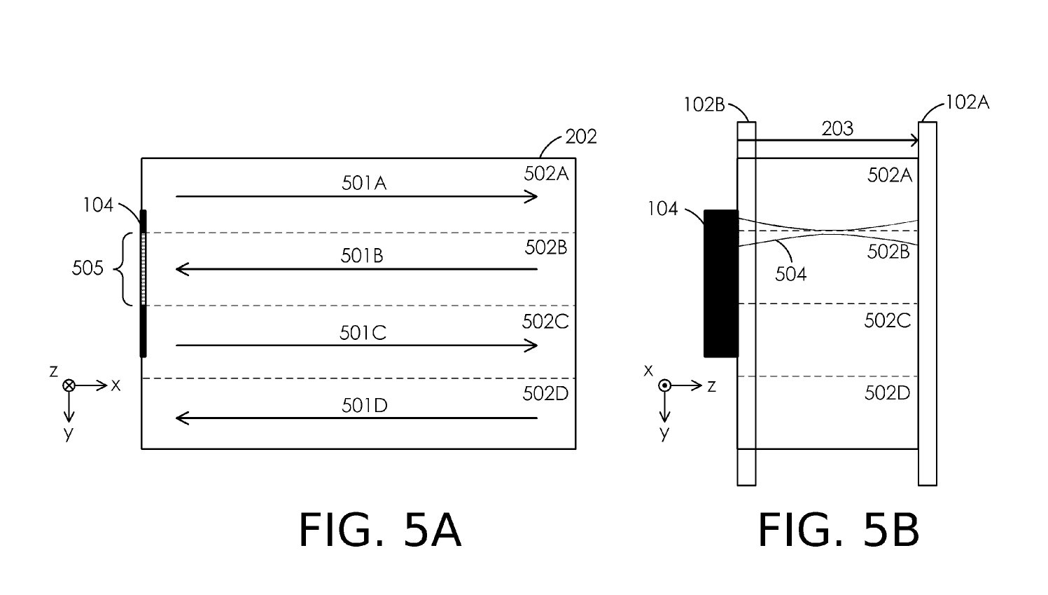

Next, the method of acquiring ultrasound signals in the reference measurement depth will be described with reference to FIG. 5. Like FIG. 2A, FIG. 5A is a front view of the held object 101 viewed from the holding plate 102B with which the probe is in contact, and FIG. 5B is a side view of the held object 101. In addition, the object 101 is not illustrated.

Reference numerals 501A, 501B, 501C, and 501D represent the moving trajectory (main scanning) of the probe in each y-axis position (the sub-scanning positions of the probe), and reference numerals 502A, 502B, 502C, and 502D represent the areas of the ultrasound images acquired at each y-axis position.

An electronic scanning width 505 represents electronic scanning width of the ultrasound beams for acquiring areas 502A to 502D. As described above, the electronic scanning width 505 may be defined by the relationship among the measurement depth (the same value as the reference measurement depth 203 in the present example), the moving speed 303 of the probe 104, and the acquisition pitch 302 of the ultrasound signal.

In the present example, since the measurement depth does not exceed the reference measurement depth, the adjustment of the number of transmitting of ultrasound beams and receiving of reflected waves and the electronic scanning width is not performed. The electronic scanning is performed by using the predetermined number of transmitting of ultrasound beams and receiving of reflected waves so that the signal acquisition time 314 does not exceed the period 311.

The moving control unit 109 orders the scanning in the determined electronic scanning width to the scanning control unit 110 to perform the electronic scanning. At the same time, the moving control unit 109 issues an order to the moving mechanism 108 to move the probe 104 in the x-axis direction and the y-axis direction. The detailed processing flow will be described below.

In the example of FIG. 5, since the determined electronic scanning width is set to be ¼ of a length in the y-axis direction of the scanning area, the scanning (main scanning) of the probe in the x-axis direction is repeated four times so as to acquire the ultrasound data.

As described above, the ultrasound measuring apparatus according to the embodiment of the present invention transmits the ultrasound beams and acquires the corresponding ultrasound signals, so as to hold the acquisition period of the ultrasound signals. The ultrasound data are generated by repeating the process at the plurality of signal acquisition start positions while moving the probe.

### Correspondence Example when Exceeding Reference Measurement Depth

Next, an example in which the depth of the measurement target exceeds the reference measurement depth will be described.

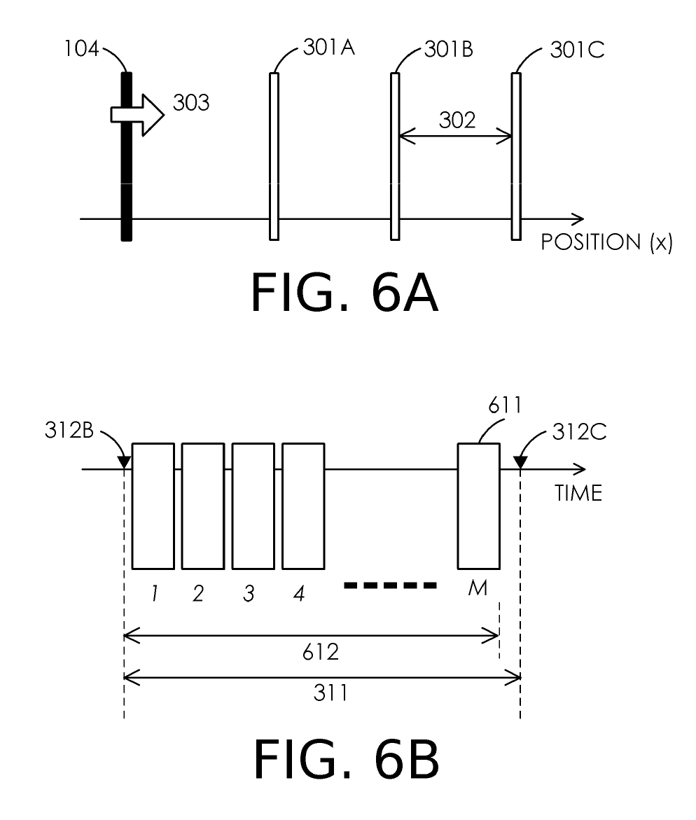

FIG. 6 is a conceptual diagram for explaining a relationship between the acquisition pitch of the ultrasound signals and the signal acquisition start time according to the electronic scanning at the time of measuring the object depth portion exceeding the reference measurement depth, in the first embodiment.

Even when intending to measure the measurement depth deeper than the reference measurement depth 203, the acquisition of the ultrasound signals needs to be completed within the time of the ultrasound signal acquisition period 311, like the case of FIG. 3.

A transmitting and receiving time 611 represents time required to transmit the ultrasound beams measuring an area deeper than the reference measurement depth once and receive the ultrasound signals, wherein a horizontal width corresponds to the transmitting and receiving time. That is, it can be appreciated that the transmitting and receiving time for measuring the object depth portion is longer as compared with FIG. 3B.

For this reason, in order to complete the acquisition of the ultrasound signals within the acquisition period 311 of the ultrasound signals, the number of ultrasound beams for performing the scanning needs to be limited to be smaller than N that is the number of original ultrasound beams, such that the signal acquisition time becomes shorter than the period 311. A method of calculating the number of ultrasound beams is the same as the foregoing method. In the case of the present example, the number of ultrasound beams is limited to M that is smaller than N that is the number of original ultrasound beams.

That is, when Equation (3) is applied with the measurement depth as D, M that is the number of ultrasound beams along the sub-scanning direction becomes a maximum integer meeting M≦(L/u)/(2D/vb).

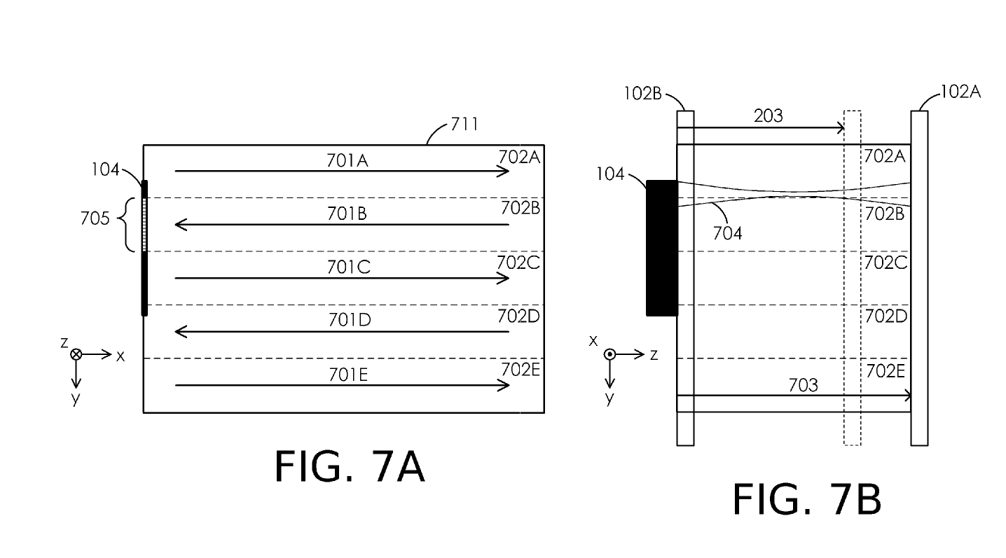

Next, the method of acquiring ultrasound signals at the time of measuring the object depth portion exceeding the reference measurement depth will be described with reference to FIG. 7. Like FIG. 5, FIG. 7A is a front view of the held object 101 viewed from the holding plate 102B with which the probe is in contact, and FIG. 7B is a side view of the held object 101.

Reference numerals 701A, 701B, 701C, 701D, and 701E represent the moving trajectory of the probe in each y-axis position (the sub-scanning positions of the probe), and reference numerals 702A, 702B, 702C, 702D, and 702E represent the areas of the ultrasound images acquired at each y-axis position.

A measurement depth 703 represents the depth for measuring the object depth portion exceeding the reference measurement depth 203 and is measured by the ultrasound beam 704 having the controlled beam shape so as to measure the measurement depth 703.

In the present example, the moving control unit 109 determines the number of transmitting of ultrasound beams and receiving of reflected waves depending on the relationship of Equations (1) and (2) and multiplies the number of transmitting of ultrasound beams and receiving of reflected waves an interval of the elements of the probe 104 to calculate the electronic scanning width.

An electronic scanning width 705 represents electronic scanning width for acquiring the ultrasound images from areas 702A to 702E. In the present example, since the number of ultrasound beams is limited, the electronic scanning width 705 is narrower than the electronic scanning width 505 as shown in FIG. 5.

That is, since the shifted amount for the y-axis direction of the probe is reduced, the ultrasound measurement of the measurement depth 703 exceeding the reference measurement depth 203 is performed, and in order to acquire ultrasound data 711, the scanning frequency of the probe in the x-axis direction needs to be increased. In the present example, since the determined electronic scanning width is ⅕ of a length in the y-axis direction of the scanning area, the scanning (main scanning) of the probe in the x-axis direction is repeated five times.

As described above, when intending to obtain the measurement depth exceeding the reference measurement depth, the processing time is secured by reducing the number of ultrasound beams at the time of performing the electronic scanning once, which is the first embodiment.

(Processing Flow Chart)

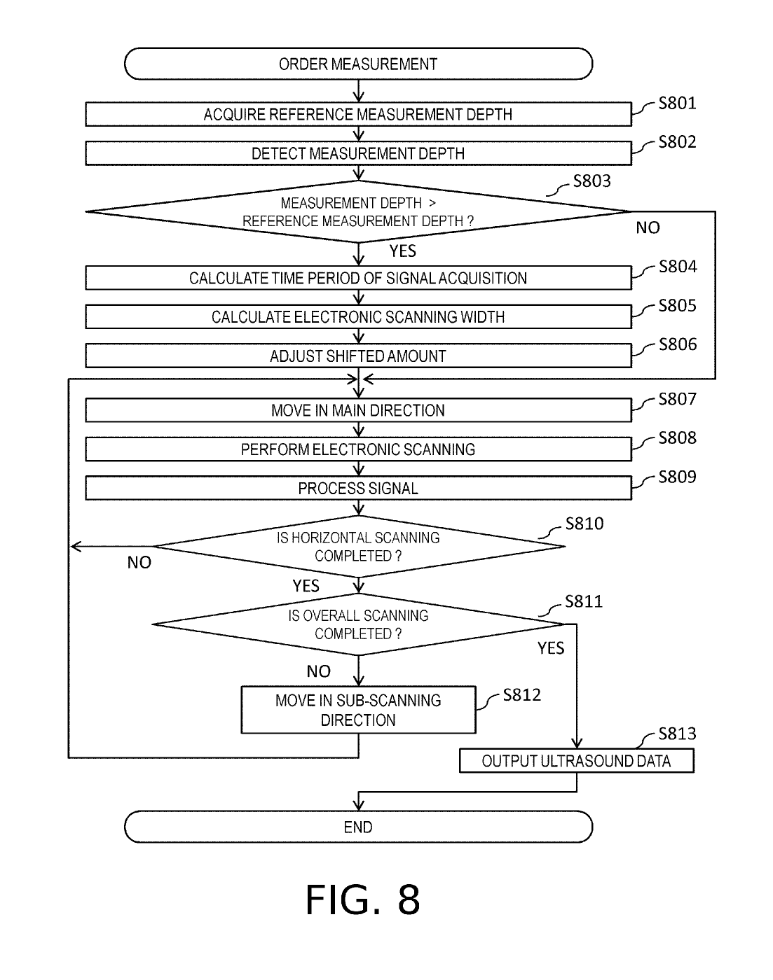

The operation of the ultrasound measuring apparatus according to the first embodiment will be described in detail with reference to FIG. 8 that is the processing flow chart of the measuring apparatus 100. Further, a measurement preparing operation such as the holding of the object, and the like, by a tester is completed before the present flow chart starts.

First, when the tester orders acquiring the ultrasound data through the operation unit 124, the ordered control unit 111 orders the moving control unit 109 to start the acquisition of the ultrasound data.

Parameters required to acquire the ultrasound images, such as the acquisition pitch of the ultrasound signals required for the targeted three-dimensional ultrasound images, and the like, are transmitted from the image processing apparatus 120 to the control unit 111, together with the start order of the ultrasound measurement from the image processing apparatus 120. Further, the parameters may be designated by the tester or may be determined from the voxel pitch of the targeted three-dimensional ultrasound images.

When the moving control unit 109 receives the acquisition start order, the moving control unit 109 acquires the reference measurement depth peculiar to the apparatus (S801) and then, receives the information including the maintenance distance of the object output from the holding control unit 103 to acquire the information on the measurement depth (S802).

Next, the moving control unit 109 determines whether the measurement depth exceeds the reference measurement depth (S803). If it is determined that the measurement depth exceeds the reference measurement depth, the process proceeds to Step 804, which performs the adjustment of the electronic scanning width and the adjustment of the shifted amount of the probe scanning. If it is determined that the measurement depth does not exceed the reference measurement depth, the process proceeds to Step 807, which performs the acquisition of the ultrasound data using the predetermined number of transmitting of ultrasound beams and receiving of reflected waves without performing the adjustment of the electronic scanning width and the adjustment of the shifted amount of the probe scanning.

When the measurement depth exceeds the reference measurement depth, the moving control unit 109 calculates the signal acquisition time required to measure the measurement depth calculated in Step S802 based on Equation (1) (S804). In addition, the signal acquisition time is calculated in consideration of the measurement depth and the sound speed within the object 101 or the holding plate 102B, but the ultrasound transmitting and receiving time may be adjusted by correcting the sound speed within the object 101 based on the holding pressure.

At the same time, the moving control unit 109 acquires the restriction time, that is, the acquisition period 311 of the ultrasound signals, based on the resolution of the ultrasound images to be generated and the moving speed of the probe. The restriction time is determined by the moving speed 303 in the main scanning direction of the probe 104 defined by Equation (2), that is, defined as one of the specifications of the apparatus and the acquisition pitch 302 of the acquired ultrasound signals.

Next, the moving control unit 109 compares the acquired restriction time with the calculated signal acquisition time to determine the number of ultrasound beams so as to meet Equation (3) and determine the electronic scanning width (S805). The processes of Steps S802 to S805 correspond to the adjusting unit in the object information acquiring apparatus to which the present invention may be applied.

Next, the moving control unit 109 adjusts the repeated number in the sub-scanning direction in the probe scanning of the probe 104 as shown in FIG. 7 based on the electronic scanning width adjusted in Step 805 (S806). When the acquisition area of the ultrasound data does not coincide with the shifted amount in the sub-scanning direction of the probe 104, the electronic scanning width may be adjusted at the final sub-scanning position. Further, the adjusted amount of the electronic scanning width at the final sub-scanning position is equivalently distributed at all the sub-scanning positions, such that all the electronic scanning widths may be adjusted to be equal. When Step S806 is completed, the moving control unit 109 starts the two-dimensional scanning by the probe 104.

In Step S807, the moving control unit 109 controls the movement of the probe 104 in the main scanning direction using the moving mechanism 108 and moves the probe 104 to the next signal acquisition start position.

When the probe 104 reaches the next signal acquisition start position, the moving control unit 109 orders the scanning control unit 110 to perform the electronic scanning in the electronic scanning width determined in Step S805 (S808).

When the electronic scanning ends, the signal processing unit 107 performs the receiving focus processing on the received ultrasound signals and writes it (S809). The information required to generate a single tomographic image is collected by performing the signal processing.

If the signal processing is completed, then the moving control unit 109 determines whether the scanning in the main scanning direction of the probe 104 is completed (S810). To determine the completion of the scanning, it is determined whether the movement in the main scanning direction for the acquisition area of the ultrasound data designated by the user is completed. When the movement is completed, the process proceeds to Step 811. Otherwise, the process proceeds to Step 807, which repeats the acquisition of the ultrasound signals at the next signal acquisition start position.

When the scanning in the main scanning direction is completed, the moving control unit 109 determines whether the overall scanning is completed for the designated ultrasound data acquisition area (S811). When the overall scanning is completed, the process proceeds to Step 813. When the overall scanning is not completed, the process proceeds to Step S812.

When the overall scanning is not completed, the moving control unit 109 controls the moving mechanism 108 to move the probe 104 in the sub-scanning direction as much as a predetermined distance and continues the acquisition operation of the ultrasound data (S812). As such, the scanning is performed by repeating the processes of Steps S807 to S812.

When the overall scanning is completed, the control unit 111 outputs the acquired ultrasound data to the image processing apparatus 120 (S813).

The acquisition of the ultrasound data exceeding the reference measurement depth may be performed by performing the foregoing processes. In addition, the processes of Steps S806 to S813 correspond to the scanning unit in the object information acquiring apparatus to which the present invention may be applied.

According to the present embodiment, in the ultrasound measuring apparatus which performs the measurement of the ultrasound waves while allowing the ultrasound probe to perform the two-dimensional scanning so as to acquire the ultrasound data, it is possible to acquire the ultrasound data of the object depth portion exceeding the reference measurement depth. That is, it is possible to resolve the problem in that the time required to perform the signal processing is insufficient.

### Second Embodiment

A second embodiment of the present invention will be described with reference to the drawings.

The feature of the second embodiment is the fact that the redundant time occurring due to the rapid completion of the acquisition of the ultrasound signals is used when the ultrasound data are acquired from an area shallower than the reference measurement depth.

In addition, in the second embodiment, the configuration (FIG. 1) of the ultrasound measuring apparatus, the scanning method (FIG. 2) of the ultrasound probe, and the detailed description (FIG. 4) of the electronic scanning method are the same as the first embodiment and therefore, the description thereof will be omitted.

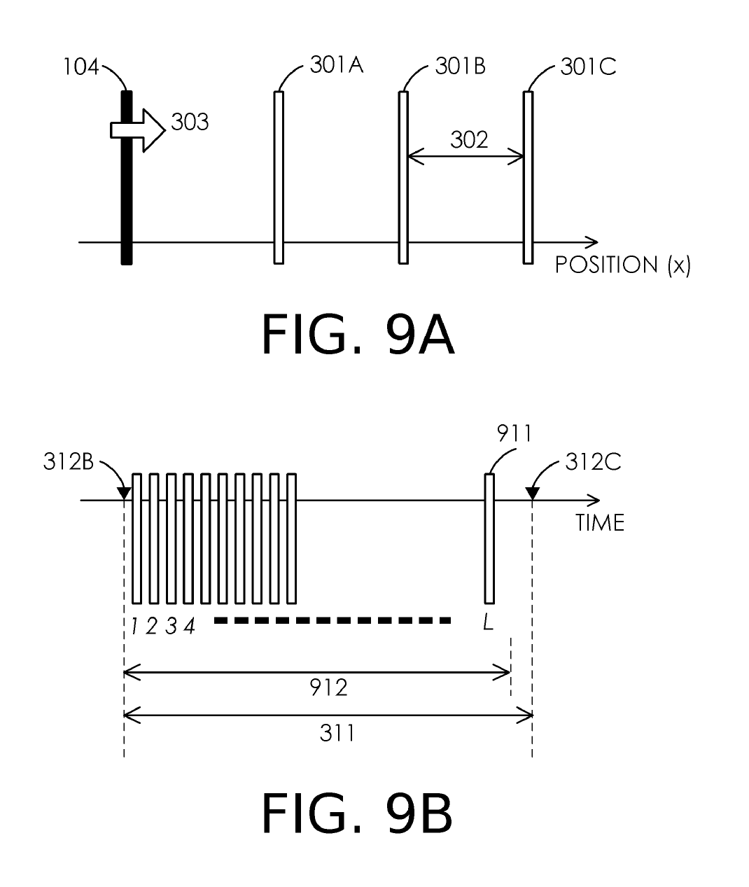

FIG. 9 is a conceptual diagram for explaining the relationship between the acquisition pitch of the ultrasound signals and the signal acquisition time according to the electronic scanning when the object of the area shallower than the reference measurement depth is measured.

Even when the measurement depth is shallower than the reference measurement depth 203, the moving speed 303 of the probe 104 and the acquisition pitch 302 of the ultrasound signals in the x-axis direction are the same. For this reason, when the acquisition of the ultrasound signals starts from the signal acquisition start position 312B, like the first embodiment, the electronic scanning may be completed up to 311 that is the acquisition period of the ultrasound signals.

A transmitting and receiving time 911 represents the time required to transmit the ultrasound beams measuring an area shallower than the reference measurement depth once and receive the ultrasound signals, wherein a horizontal width corresponds to the transmitting and receiving time. When the shallow area is measured, the transmitting and receiving time of the ultrasound signals is short and therefore, when the same number of ultrasound beams are transmitted and received, the consumed time is shorter than the transmitting and receiving time 313.

That is, it is possible to transmit and receive the ultrasound beams more than the case in which the reference measurement depth is measured, within the acquisition period 311 of the ultrasound signals. The method of calculating the number of ultrasound beams is the same as the foregoing method. In the case of the second embodiment, the number of ultrasound beams may be set to be L that is larger than N which is the number of original ultrasound beams.

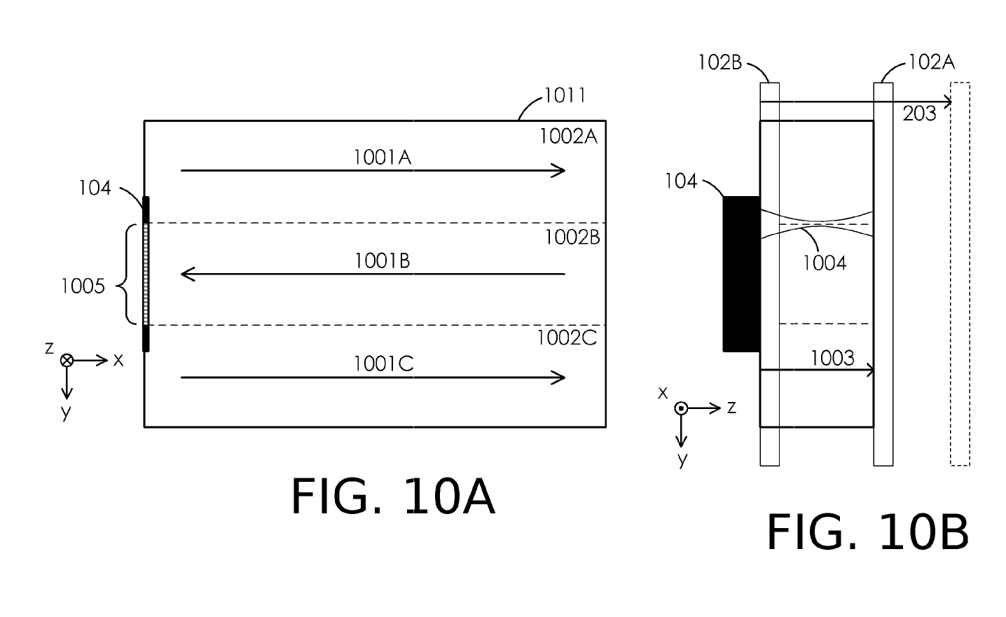

Next, the method of acquiring ultrasound data according to the second embodiment will be described with reference to FIG. 10. FIG. 10A is a front view of the held object 101 viewed from the holding plate 102B with which the probe is in contact, and FIG. 10B is a side view of the held object 101.

Reference numerals 1001A, 1001B, and 1001C represent the moving trajectory of the probe in the sub-scanning positions (y-axis positions) of the probe, and reference numerals 1002A, 1002B, and 1002C represent the areas of the ultrasound images acquired at each sub-scanning position.

Reference numeral 1003 represents the depth shallower than the reference measurement depth 203, and the measurement is performed by an ultrasound beam 1004 having the appropriately controlled beam shape.

Even in the present example, the moving control unit 109 determines the number of transmitting of ultrasound beams and receiving of reflected waves depending on the relationship between Equations (1) and (2) and multiplies the number of transmitting of ultrasound beams and receiving of reflected waves by the interval of the elements of the probe 104 to calculate the electronic scanning width. An electronic scanning width 1005 represents the electronic scanning width of the ultrasound beams for acquiring areas 702A to 702E. In the present example, since the number of ultrasound beams may be increased as compared with the case in which the reference measurement depth is measured, the electronic scanning width is larger than the electronic scanning width 505 of FIG. 5.

Therefore, when performing the ultrasound measurement of the measurement depth 1003 shallower than the reference measurement depth 203, the electronic scanning width in the y-axis direction is increased and therefore, the scanning in the x-axis direction ends by being performed less times. In the present example, since the determined electronic scanning width is ⅓ of a length in the y-axis direction of the scanning area, the scanning (main scanning) of the probe in the x-axis direction is repeated three times.

The operation of the ultrasound measuring apparatus according to the second embodiment will be described with reference to the flow chart of FIG. 8. The processes up to Step S802 are the same as the first embodiment.

In the second embodiment, the process of Step 804 is performed without performing the comparison determination in Step S803, and the determination of the electronic scanning width and the adjustment of the repeated number in the sub-scanning direction in the probe scanning are performed.

The processes after Step S804 are the same as the first embodiment.

As described with reference to FIGS. 9 and 10, when the measurement depth is shallow, the time of the acquisition period 311 of the ultrasound signals may be used as maximally as possible and the acquisition time of the ultrasound data may be wholly shortened, by making the electronic scanning width larger than a standard and adjusting the scanning trajectory of the probe.

Further, in the first embodiment, the electronic scanning width is configured so as to be maximal when the reference measurement depth is measured, but in the present embodiment, it is preferable to increase the elements and make the electronic scanning width larger than the case in which the reference measurement depth is measured.

According to the present embodiment, in the ultrasound measuring apparatus performing the measurement of the ultrasound waves while allowing the ultrasound probe to perform two-dimensionally scanning, the electronic scanning width may be increased when the measurement depth is shallower than the estimated depth. As a result, it is possible to increase the reading width per scanning and shorten the overall acquisition time of the ultrasound data.

In addition, even though the present embodiment describes the action and effect of the case in which the measurement depth is shallower than the estimated depth, the measurement depth may be deeper than the estimated depth. At any rate, the number of ultrasound beams may be determined using Equation (3) at all times without using the reference measurement depth.

The above embodiments are only examples and therefore, the present invention may be practiced by being appropriately changed without departing from the subject matters of the present invention.

For example, the illustrated embodiments adjust the number of ultrasound beams by changing the electronic scanning width, but the electronic scanning width may be held by, for example, the method of reducing ultrasound beams, that is, degrading the resolution of the ultrasound images.

Further, the object information acquiring apparatus to which the present invention may be applied may include a central processing unit (CPU) and a main storage apparatus (RAM), and an auxiliary storage apparatus (storage medium) so as to realize the function of the foregoing embodiments. When being configured as described above, a program code corresponding to the foregoing flow chart is stored in the auxiliary storage apparatus and is read and executed by the CPU, thereby realizing the function of the foregoing embodiments. In this case, an operating system (OS), and the like, that is executed on computer may perform a part or all of the processings to realize the function of the foregoing embodiments.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-246414, filed on Nov. 10, 2011, which is hereby incorporated by reference herein its entirety.

What is claimed is:

1. An object information acquiring apparatus comprising:
a probe including a plurality of elements arranged along at least a first direction and configured to sequentially perform transmitting of acoustic wave beams and receiving of reflected waves reflected from an inside of an object along the first direction by a part of or all of the elements;
a scanning unit configured to set a second direction intersecting the first direction as a main scanning direction and move the probe in the main scanning direction at a predetermined speed; and
an adjusting unit configured to acquire information on a measurement depth for acquiring object information in a transmitting direction of the acoustic wave beams and determine the number of times of transmitting of acoustic wave beams and receiving of reflected waves along the first direction based on the measurement depth, resolution of the object information in the main scanning direction, and a moving speed of the probe.

2. The object information acquiring apparatus according to claim 1, wherein the adjusting unit determines the number of times of transmitting of acoustic wave beams and receiving of reflected waves along the first direction so that a second time that is a sum of times for the plurality of times of transmitting of acoustic wave beams and receiving of the reflected waves based on the measurement depth is shorter than a first time that is determined based on the resolution of the object information to be acquired in the main scanning direction and the moving speed of the probe.

3. The object information acquiring apparatus according to claim 1, wherein the scanning unit sets the first direction as a sub-scanning direction, is able to further move the probe in the sub-scanning direction, and repeatedly moves the probe in the main scanning direction and the sub-scanning direction for acquiring information of the object.

4. The object information acquiring apparatus according to claim 3, wherein when the second time exceeds the first time in a case in which a reference number of times of transmitting of acoustic wave beams and receiving of reflected waves is used, the adjusting unit makes the number of times of transmitting of acoustic wave beams and receiving of reflected waves along the first direction smaller than the reference number of times, and
the scanning unit reduces a shifted amount of the probe in the sub-scanning direction.

5. The object information acquiring apparatus according to claim 3, wherein when the second time is smaller than the first time in a case in which a reference number of times of transmitting acoustic wave beams and receiving reflected waves is used,
the adjusting unit makes the number of times of transmitting of acoustic wave beams and receiving of reflected waves along the first direction of acoustic wave beams larger than the reference number of acoustic wave beams, and
the scanning unit increases a shifted amount of the probe in the sub-scanning direction.

6. A control method of an object information acquiring apparatus that includes an probe including a plurality of elements arranged along at least a first direction and configured to sequentially perform transmitting of acoustic wave beams and receiving of reflected waves reflected from an inside of an object along the first direction by a part of or all of the elements, and that is configured to set a second direction intersecting the first direction as a main scanning direction and move the probe in the main scanning direction at a predetermined speed to acquire object information, the method comprising the steps of:
acquiring information on a measurement depth for acquiring the object information in a transmitting direction of the acoustic wave beams; and
determining the number of times of transmitting of acoustic wave beams and receiving of reflected waves along the first direction based on the measurement depth, resolution of the object information in the main scanning direction, and a moving speed of the probe.
